(19) United States
(12) Patent Application Publication
(10) Pub. No.: US 2025/0044291 A1
(43) Pub. Date: Feb. 6, 2025

(54) METHOD FOR THE DETECTION OF BLOOD CANCER

(71) Applicant: Belgian Volition SRL, Isnes (BE)

(72) Inventors: Priscilla VAN DEN ACKERVEKEN, Isnes (BE); Marielle HERZOG, Isnes (BE)

(21) Appl. No.: 18/717,655

(22) PCT Filed: Dec. 9, 2022
(86) PCT No.: PCT/EP2022/085223

§ 371 Date: Jun. 7, 2024

(30) Foreign Application Priority Data
Dec. 9, 2021 (GB) 2117799.3

(51) Int. Cl. G01N 33/574

(52) U.S. Cl. CPC G01N 33/57407; G01N 33/57488

(57) ABSTRACT

The invention relates to methods and uses of nucleosomes comprising one or more histone modifications as a biomarker in a body fluid sample for diagnosing and/or monitoring blood cancer.

### FIELD OF THE INVENTION

The invention relates to a body fluid test method for the detection of blood cancer using a combination biomarker panel.

### BACKGROUND OF THE INVENTION

Cancer is a common disease with a high mortality. The biology of the disease is understood to involve a progression from a pre-cancerous state leading to stage I, II, III and eventually stage IV cancer. For the majority of cancer diseases, mortality varies greatly depending on whether the disease is detected at an early stage, when effective treatment options are available, or at a late stage when the disease may have spread within the organ affected or beyond when treatment is more difficult. Late stage cancer symptoms are varied including visible blood in the stool, blood in the urine, blood discharged with coughing, blood discharged from the vagina, unexplained weight loss, persistent unexplained lumps (e.g. in the breast), indigestion, difficulty in swallowing, changes to warts or moles as well as many other possible symptoms depending on the cancer type. However, most cancers diagnosed due to such symptoms will already be late stage and difficult to treat. Most cancers are symptomless at early stage or present with non-specific symptoms that do not help diagnosis. Cancer should ideally therefore be detected early using cancer tests.

There are over 544,000 new cases of Non-Hodgkin Lymphoma (NHL) diagnosed worldwide each year and approximately 260,000 deaths. The non-specific symptoms of lymphoma often delay diagnosis. Alterations of epigenetic modifications have been demonstrated as an important player in human cancer including lymphoma. However, the epigenetic profile of histone post-translational modifications (PTMs) on circulating nucleosomes is still not well described.

Circulating cell free nucleosomes containing particular epigenetic signals including particular post-translational modifications, histone isoforms, modified nucleotides and non-histone chromatin proteins have also been investigated as markers of cancer (as referenced in WO2005019826, WO2013030577, WO2013030579 and WO2013084002). Cell free nucleosomes have also been described as biomarkers in plasma samples for vascular or haematological cancers (WO2021110776).

Despite recent advances, very few blood test methods are used routinely during cancer screening. There is a need to develop non-invasive blood tests for the management and guidance of cancer treatment and for cancer detection and diagnosis, to rule cancer in or out as a potential diagnosis in symptomatic patients or as an adjunct to other cancer detection methods.

### SUMMARY OF THE INVENTION

According to a first aspect of the invention, there is provided the use of a nucleosome comprising one or more histone modifications selected from the group consisting of: H3K9Ac, H3K9Me, H3K14Ac, H3K18Ac, H3K23Ac, H3K27Me2, H3K27Me3, H3K36Me, H3K36Me2 and H3K36Me3, as a biomarker, in a body fluid sample for diagnosing and/or monitoring blood cancer.

According to a further aspect of the invention, there is provided a method of diagnosing blood cancer in a patient, comprising:
detecting or measuring the level of one or more nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3, in a body fluid sample obtained from the patient; and
using the level detected in the body fluid sample to determine if the patient has blood cancer.

According to a further aspect of the invention, there is provided a method of treating blood cancer in a patient, comprising;
(i) detecting or measuring the level of one or more nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3, in a body fluid sample obtained from the patient;
(ii) using the level detected in the body fluid sample to determine if the patient has blood cancer; and
(iii) administering a treatment to the patient if they are determined to have blood cancer in step (ii).

According to a further aspect of the invention, there is provided a kit comprising reagents to detect two or more nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3.

### BRIEF DESCRIPTION OF THE FIGURES

FIG. 1: Enrichment of nucleosomes in blood samples. (A) There is a higher level of circulating nucleosomes in Non-Hodgkin Lymphoma (NHLym) patients. Box plots showing the concentration of circulating H3.1-nucleosomes (ng/ml) quantified by anti-H3.1 antibody immunoassay from plasma samples of healthy donors (n=5) or NHLym patients (n=9). Box plot shows mean value and 25th and 75th percentiles, the whiskers represent the standard deviation (Mann-Whitney test; p-Value=0.0599). (B) Results showing the depletion of nucleosomes after anti-H3.1 antibody immunoprecipitation (light grey square dots) in comparison to the level present in the initial plasma (black circular dots). The protocol was able to isolate 92.7%±5.3% of the nucleosomes present in the plasma sample of NHLym patients.

FIG. 2: Quantitative analysis of histone post-translational modifications (PTMs) from circulating nucleosomes by LC-MS/MS. Heat map showing the histone PTMs peptides identified in plasma samples from NHLym patients (n=9; from #1 to #9) and Healthy donors (n=6; from #10 to #15). Using the mass spectrometry protocol described in Example 1, 56 histone proteoforms were identified. Core histone proteins such as histone H3 and H4 were identified in all immunoprecipitated samples confirming that the immunoprecipitation protocol isolates circulating nucleosomes. Data are shown as Log 2 Intensity of histone PTM levels.

FIG. 3: Histone PTMs on circulating nucleosomes differentially represented in NHLym patients. Box plots showing the abundance (Log 2 Intensity) of 5 histone peptides, representing 7 histone PTMs (as two of the peptides contain 2 PTMs), differentially abundant in plasma from NHLym patients vs Healthy donors: (A) H3_K27Me2_K36Me2; (B) H3.1_K9Ac_K14Ac; (C) H3_K9Me1; (D) H3_K23Ac; and (E) H3.1_K18Ac. The box plot shows the median and the 25th and 75th percentiles; the whiskers indicate the 5th and 95th percentiles.

FIG. 4: H3.1-nucleosomes are associated with cell free circulating DNA (cfDNA) in NHL patients. (a) Box plot showing the increased cfDNA concentration (ng/ml) in plasma from NHL patients (n=7) compared to healthy donors (n=5) (*: p=0.037). (b) cfDNA Fragment size distribution of a representative Healthy donor compared to NHL patient using Agilent 2100 Bioanalyzer. (c) Box plot showing significant increase of the H3.1-nucleosome concentration (ng/ml) from the NHL patients (n=9) compared to the healthy donor (n=5) (*: p=0.012).

FIG. 5: Mass spectrometry allows the epigenetic profile analysis of circulating nucleosomes of NHL patients. Box plot showing the differential abundance of histone peptides detected by mass spectrometry using the method described in Example 1, in plasma sample from healthy donors (n=5) compared to NHL patients (n=9). Results are expressed as log 2 (ratio) of histone PTMs levels.

FIG. 6: Histone H3 PTMs measured by immunoassay. Box plots showing quantifications by immunoassays of (a) circulating nucleosome H3.1 (***: p<0.001), (b) H3K9Ac (***: p<0.001), (c) H3K14Ac (**: p=0.001), (d) H3K18Ac (*: p=0.022), (e) H3K9Me1 (***: p<0.001), (f) H3K27Me3 (labelled as “H3K27Methyl”) (***: p<0.001), (g) H3K36Me3 (labelled as “H3K36Methyl”) (***: p<0.001), from NHL patients (n=24) compared to healthy donors (n=34). p-values were determined by Mann-Whitney.

### DETAILED DESCRIPTION OF THE INVENTION

According to a first aspect of the invention, there is provided the use of a nucleosome comprising one or more histone modifications selected from the group consisting of: H3K9Ac, H3K9Me, H3K14Ac, H3K18Ac, H3K23Ac, H3K27Me2, H3K27Me3, H3K36Me, H3K36Me2 and H3K36Me3 as a biomarker, in a body fluid sample for diagnosing and/or monitoring blood cancer. Data is presented herein that shows that this selection of biomarkers was able to discriminate between patients with blood cancer compared to healthy controls.

According to another aspect of the invention, there is provided the use of a nucleosome comprising one or more histone modifications selected from the group consisting of: H3K9Ac, H3K9Me, H3K14Ac, H3K18Ac, H3K23Ac, H3K27Me2 and H3K36Me2 as a biomarker, in a body fluid sample for diagnosing and/or monitoring blood cancer. Data is presented herein that shows that this selection of biomarkers was able to discriminate between patients with blood cancer compared to healthy controls.

The selected biomarkers comprise the histone post-translational modifications (PTMs): H3K9Ac (i.e. acetylated histone H3 at the lysine residue in position 9), H3K9Me (i.e. methylated histone H3 at the lysine residue in position 9), H3K14Ac (i.e. acetylated histone H3 at the lysine residue in position 14), H3K18Ac (i.e. acetylated histone H3 at the lysine residue in position 18), H3K23Ac (i.e. acetylated histone H3 at the lysine residue in position 23), H3K27Me2 (i.e. di-methylated histone H3 at the lysine residue in position 27), H3K27Me3 (i.e. tri-methylated histone H3 at the lysine residue in position 27), H3K36Me (i.e. mono-methylated histone H3 at the lysine residue in position 36), H3K36Me2 (i.e. di-methylated histone H3 at the lysine residue in position 36) and H3K36Me3 (i.e. tri-methylated histone H3 at the lysine residue in position 36). As the biomarkers described herein refer to histone PTMs present in nucleosomes, said biomarkers may also be referred to as “nucleosome biomarkers”. Therefore, according to an aspect of the invention, there is provided the use of one or more nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3, in a body fluid sample for diagnosing and/or monitoring blood cancer. According to another aspect of the invention, there is provided the use of one or more nucleosome biomarkers selected from the group consisting of: H3K9Ac, H3K9Me, H3K14Ac, H3K18Ac, H3K23Ac, H3K27Me2 and H3K36Me2, in a body fluid sample for diagnosing and/or monitoring blood cancer.

As described herein, one or more of the listed biomarkers may be detected. Therefore, two or more, three or more, four or more, five or more, six or more, seven or more, eight or more, nine or more or all ten of the biomarkers may be detected. In one embodiment, the use comprises a panel of two or more of said biomarkers. In a further embodiment, the panel comprises two or more, three or more, four or more, five or more, six or more, seven or more, eight or more, nine or more or all ten of the biomarkers.

In one embodiment, the one or more (e.g. two or more, or all) histone modifications are selected from the group consisting of: H3K9Ac, H3K9Me, H3K14Ac, H3K18Ac, H3K23Ac, H3K27Me3 and H3K36Me3. In a further embodiment, H3K9Ac, H3K9Me, H3K14Ac, H3K18Ac, H3K23Ac, H3K27Me3 and H3K36Me3 are detected.

In one embodiment, the one or more (e.g. two or more, or all) histone modifications are selected from the group consisting of: H3K9Ac, H3K9Me, H3K14Ac, H3K18Ac, H3K27Me3 and H3K36Me3. In a further embodiment, H3K9Ac, H3K9Me, H3K14Ac, H3K18Ac, H3K27Me3 and H3K36Me3 are detected.

In one embodiment, the one or more (e.g. two or more, or all) histone modifications are selected from the group consisting of: H3K9Ac, H3K9Me, H3K14Ac, H3K18Ac, H3K23Ac, H3K36Me, H3K36Me2 and H3K36Me3. In a further embodiment, H3K9Ac, H3K9Me, H3K14Ac, H3K18Ac, H3K23Ac, H3K36Me, H3K36Me2 and H3K36Me3 are detected.

According to any of the aspects described herein, the one or more histone modifications are selected from the group consisting of: H3K9Ac, H3K9Me, H3K14Ac, H3K18Ac, H3K23Ac, H3K27Me2 and H3K36Me2.

In one embodiment, the biomarkers comprise: H3K9Ac and H3K14Ac. In another embodiment, the biomarkers comprise: H3K27Me2 and H3K36Me2.

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller (2007)).

In one embodiment, the nucleosome is a cell free nucleosome. References to “nucleosome” may refer to “cell free nucleosome” when detected in body fluid samples. It will be appreciated that the term “cell free nucleosome” used throughout this document is intended to include any cell free chromatin fragment that includes one or more nucleosomes.

In one embodiment, the use and methods described herein comprise one or more additional biomarkers. For example, additional biomarkers may also comprise cell free nucleosomes per se, one or more epigenetic features of cell free nucleosomes and the level of cell free DNA. Epigenetic signal structures/features of a cell free nucleosome as referred herein may comprise, without limitation, one or more histone post-translational modifications, histone isoforms/variants, modified nucleotides and/or proteins bound to a nucleosome as a nucleosome-protein adduct.

References to “nucleosomes per se” refers to the total nucleosome level or concentration present in the sample, regardless of any epigenetic features the nucleosomes may or may not include. This type of assay is also often referred to simply as a “nucleosome assay” or as a “total nucleosome assay” and typically involves detecting a histone protein common to all nucleosomes, such as histone H4 or histone H3. Therefore, in one embodiment, nucleosomes per se are measured by detecting a core histone protein, such as histone H3. As described herein, histone proteins form structural units known as nucleosomes which are used to package DNA in eukaryotic cells. In one embodiment, the histone protein is a core histone, such as H2A, H2B, H3 or H4. As previously reported in WO2016067029 (incorporated herein by reference), particular histone variants, such as histone H3.1, H3.2 or H3t, may be used to isolate cell free nucleosomes originating from tumour cells.

Mononucleosomes and oligonucleosomes can be detected by Enzyme-Linked ImmunoSorbant Assay (ELISA) and several methods have been reported (Salgame et al, 1997; Holdenrieder et al, 2001; van Nieuwenhuijze et al, 2003; WO2005019826; WO2013030577; WO2013030579; and WO2013084002, all of which are herein incorporated by reference). These assays typically employ an anti-histone antibody (for example anti-H2B, anti-H3 or anti-H1, H2A, H2B, H3 and H4) as capture antibody and detection antibody (which varies depending upon the moiety to be detected) or an anti-histone antibody as capture antibody and an anti-DNA antibody as detection antibody. In one embodiment, the anti-histone antibody comprises an anti-H3 antibody or an anti-H1 antibody. In particular, the anti-histone antibody may comprise an anti-H3.1 antibody, for example as described in WO2016067029.

Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in nucleosome levels will be associated with elevations in some circulating nucleosome subsets containing particular epigenetic signals including nucleosomes comprising particular histone isoforms (or variants), comprising particular post-translational histone modifications, comprising particular nucleotides or modified nucleotides and comprising particular protein adducts. Assays for these types of chromatin fragments are known in the art (for example, see WO2005019826, WO2013030579, WO2013030578, WO2013084002 which are herein incorporated by reference).

In one embodiment, the additional biomarker is selected from a histone post-translational modification, histone isoform, modified nucleotide and/or a protein bound to a nucleosome (i.e. as a nucleosome-protein adduct).

The structure of a nucleosome may vary by the inclusion of alternative histone isoforms or variants which are different gene or splice products and have different amino acid sequences. Therefore, in one embodiment, the additional biomarker comprises a histone isoform. Many histone isoforms are known in the art. The nucleotide sequences of a large number of histone isoforms are publicly available for example in the National Human Genome Research Institute NHGRI Histone DataBase (Mariño-Ramirez et al. The Histone Database: an integrated resource for histones and histone fold-containing proteins. Database Vol. 2011), the GenBank (NIH genetic sequence) DataBase, the EMBL Nucleotide Sequence Database and the DNA Data Bank of Japan (DDBJ). In one embodiment, the additional biomarker is a histone isoform, such as a histone isoform of a core histone. In a preferred embodiment, the additional biomarker comprises a histone isoform of histone H3, for example a histone isoform selected from H3.1, H3.2 and H3t, in particular H3.1.

Another way the structure of nucleosomes may vary is by mutation. Therefore, in one embodiment, the additional biomarker is a mutated histone. In a further embodiment, the mutation is in histone 3 (H3). In a yet further embodiment, the mutation in H3 is when lysine 27 is replaced by a methionine (H3K27M).

In another embodiment, the additional biomarker comprises one or more further post-translational histone modifications. As described herein, the structure of nucleosomes can vary by post translational modification (PTM) of histone proteins. PTM of histone proteins typically occurs on the tails of the core histones and common modifications include acetylation, methylation or ubiquitination of lysine residues as well as methylation of arginine residues and phosphorylation of serine residues and many others. Many histone modifications are known in the art and the number is increasing as new modifications are identified (Zhao and Garcia, 2015). Examples of PTMs are described in WO 2005/019826 and WO 2017/068359.

For example, the post translational modification may include acetylation, methylation, which may be mono-, di- or tri-methylation, phosphorylation, ribosylation, citrullination, ubiquitination, hydroxylation, glycosylation, nitrosylation, glutamination and/or isomerisation (see Ausio (2001) Biochem Cell Bio 79:693). In one embodiment, the histone PTM is methylation, in particular methylation (in particular, di-methylation or tri-methylation) of a lysine residue. For the avoidance of doubt, references herein to mono-methylation may also be referred to as “Me” or “Me1”; references herein to di-methylation may also be referred to as “Me2”; and references herein to tri-methylation may as be referred to as “Me3”.

In one embodiment, a group or class of related histone (post translational) modifications (rather than a single modification) is detected. A typical example of this embodiment, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes without limitation, anti-pan-acetylation antibodies (e.g. a Pan-acetyl H4 antibody), anti-citrullination antibodies or anti-ubiquitin antibodies.

In one embodiment, the additional biomarker comprises one or more DNA modifications (i.e. modified nucleotides). In addition to the epigenetic signalling mediated by nucleosome histone isoform and histone post-translational modification composition, nucleosomes also differ in their nucleotide and modified nucleotide composition. Global DNA hypomethylation is a hallmark of cancer cells and some nucleosomes may comprise more 5-methylcytosine residues (or 5-hydroxymethylcytosine residues or other nucleotides or modified nucleotides) than other nucleosomes. 5-hydroxymethylation may be detected, for example, at CpG islands in the genome. In one embodiment, the DNA modification is selected from 5-methylcytosine or 5-hydroxymethylcytosine.

In another embodiment, the additional biomarker comprises a protein adduct, i.e. a nucleosome and another non-histone protein which is adducted to the nucleosome or chromatin fragment. Such adducts may include any protein that contains or includes a DNA binding domain or a nucleosome binding domain or a histone binding domain. Examples include transcriptions factors, structural chromatin proteins, CpG methyl-CpG binding domain proteins, high mobility group box proteins (e.g. HMGB1), epigenetic enzymes such as histone acetyl transferases, histone methyl transferases, histone deacetylases, DNA methyltransferases, PARP (poly-ADP ribose polymerase) binders and many others.

In one embodiment, the protein adducted to the nucleosome (and which therefore may be used as a biomarker) is selected from: a transcription factor, a High Mobility Group Protein or chromatin modifying enzyme. References to “transcription factor” refer to proteins that bind to DNA and regulate gene expression by promoting (i.e. activators) or suppressing (i.e. repressors) transcription. Transcription factors contain one or more DNA-binding domains (DBDs), which attach to specific sequences of DNA adjacent to the genes that they regulate. All of the circulating nucleosomes and nucleosome moieties, types or subgroups described herein may be useful in the present invention.

In one embodiment, the additional biomarker comprises the level of cell free DNA (cfDNA). As shown by the Examples presented herein, the level of cfDNA correlated well with the level of nucleosomes present in the sample. Therefore, an elevated level of cfDNA could also be used as a marker for a blood cancer. The level of cfDNA in a sample can be measured using methods known in the art, for example by extracting DNA from the sample (e.g. using QIAamp® DSP Circulating NA kit from Qiagen) and measuring using a DNA assay (e.g. Qubit DNA Assay from ThermoFisher).

The sample may be any biological fluid (or body fluid) sample taken from a subject including, without limitation, cerebrospinal fluid (CSF), whole blood, blood serum, plasma, menstrual blood, endometrial fluid, urine, saliva, or other bodily fluid (stool, tear fluid, synovial fluid, sputum), breath, e.g. as condensed breath, or an extract or purification therefrom, or dilution thereof. In a preferred embodiment, the body fluid sample is selected from blood, serum or plasma. Biological samples also include specimens from a live subject, or taken post-mortem. The samples can be prepared, for example where appropriate diluted or concentrated, and stored in the usual manner. It will be understood that methods and uses of the present invention find particular use in blood, serum or plasma samples obtained from a patient. In one embodiment, the sample is a blood or plasma sample, in particular a plasma sample. In a further embodiment, the sample is a serum sample.

According to a further aspect of the invention, there is provided the use of one or more binding agents in the manufacture of a kit for diagnosing and/or monitoring blood cancer in a body fluid sample, wherein the binding agents detect one or more nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3.

### Blood Cancer

Blood cancers, which may also be referred to as “haematological cancers”, are the types of cancer affecting blood, bone marrow and lymph nodes. They are referred to as leukaemia, lymphoma and myeloma depending on the type of blood cell affected. Leukaemia is cancer of the blood cells which usually starts in the bone marrow and travels through the bloodstream. In leukaemia, the bone marrow produces mutated cells and spreads them into the blood, where they grow and crowd out healthy blood cells. Lymphoma diseases affect the cells in the lymphatic system. In lymphomas, immune cells called lymphocytes grow out of control and collect in lymph nodes, the spleen, in other lymph tissues or in neighbouring organs. Myeloma, also known as multiple myeloma, develops in the bone marrow and affects plasma cells, which produce antibodies that attack infections and diseases. Examples of blood cancers include Acute Lymphoblastic Leukaemia (ALL), Acute Myeloid Leukaemia (AML), Hodgkin Lymphoma (HL) and Non-Hodgkin Lymphoma (NHL, or may also be referred to as “NHLym”).

HL and NHL are the two main types of lymphoma. HL has a particular appearance under the microscope and contains cells called Reed-Sternberg cells (a type of B lymphocyte that has become cancerous), whereas NHL looks different under the microscope and does not contain Reed-Sternberg cells. Most lymphomas are NHL and only about 1 in 5 are HL. NHL is a cancer affecting lymphocytes and usually starts in lymph nodes or lymph tissue. NHL is typically not diagnosed until it has reached a more advanced stage, therefore methods of the invention find particular use in the diagnosis of NHL where there is a need to detect patients at an early stage of disease to improve treatment outcome.

Current methods of diagnosing leukaemia and myeloma involve obtaining a complete blood count (CBC) test to identify abnormal levels of white blood cells relative to red blood cells and platelets. However, an elevated white blood cell count (WBC) is not specific to patients with a haematological malignancy; it can also be the result of an ongoing response to infection or other inflammatory process. For lymphoma, an X-ray, CT or PET scan can be used to detect swollen lymph nodes, however this is also non-specific.

In order to confirm a diagnosis of a haematological cancer, a bone marrow or lymph node biopsy is required. Therefore overdiagnosis of haematological cancers at an early stage in the diagnostic process can lead to unnecessary biopsies which are invasive, potentially hazardous and relatively costly to healthcare providers. Cytogenetics analysis and/or immunophenotyping can also be used to confirm a haematological cancer diagnosis, however these methods are expensive to perform and therefore are typically only used at a late stage of the diagnostic process.

In one embodiment the blood cancer is selected from lymphoma, leukaemia, myeloma, chronic myeloproliferative disease, monoclonal gammopathy of uncertain significance, myelodysplastic syndrome and amyloidosis. In a further embodiment, the haematological cancer is selected from lymphoma. In a further embodiment, the lymphoma is Non-Hodgkin Lymphoma (NHL). NHL is a common type of haematological malignant tumour that mainly originates from lymphocytes.

### Diagnosis and Treatment Methods

According to a further aspect of the invention, there is provided a method of diagnosing blood cancer in a patient, comprising:
detecting or measuring the level of one or more nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3, in a body fluid sample obtained from the patient; and
using the level detected in the body fluid sample to determine if the patient has blood cancer.

In one embodiment, the method additionally comprises determining at least one clinical parameter for the patient. This parameter can be used in the interpretation of results. Clinical parameters may include any relevant clinical information for example, without limitation, gender, weight, Body Mass Index (BMI), smoking status and dietary habits. Therefore, in one embodiment, the clinical parameter is selected from the group consisting of: family history of blood cancer, age, sex and body mass index (BMI).

In one embodiment individual assay cut-off levels are used and the patient is considered positive in the panel test if individual panel assay results are above (or below if applicable) the assay cut-off level for all or a minimum number of the panel assays (for example, one of two, two of two, two of three etc). In one embodiment of the invention a decision tree model or algorithm is employed for analysis of the results.

It will be clear to those skilled in the art, that any combination of the nucleosome biomarkers disclosed herein may be used in panels and algorithms for the detection of cancer and that further markers may be added to a panel including these markers.

According to a further aspect of the invention, there is provided a method of treating blood cancer in a patient, comprising;
(i) detecting or measuring the level of one or more nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3, in a body fluid sample obtained from the patient;
(ii) using the level detected in the body fluid sample to determine if the patient has blood cancer; and
(iii) administering a treatment to the patient if they are determined to have blood cancer in step (ii).

Treatments available for blood cancer include hormone therapy, immunotherapy, as well as a variety of drug treatments for use in chemotherapy. In one embodiment, the treatment(s) administered are selected from: chemotherapy, immunotherapy, hormone therapy, biological therapy, radiotherapy, leukapheresis and stem cell transplant.

According to another aspect of the invention there is provided a method of treatment for blood cancer comprising identifying a patient in need of treatment for blood cancer by detecting one or more nucleosome biomarkers of the invention in a body fluid sample obtained from the patient and providing said treatment, wherein the biomarkers are selected from the group consisting of: H3K9Ac, H3K9Me, H3K14Ac, H3K18Ac, H3K23Ac, H3K27Me2, H3K27Me3, H3K36Me, H3K36Me2 and H3K36Me3.

In one embodiment, patients are at high risk for blood cancer if they have elevated levels of one or more said nucleosome biomarkers compared to a control.

In one embodiment, the control comprises a healthy subject, a non-diseased subject and/or a subject without cancer. In one embodiment, the method comprises comparing the amount of nucleosome biomarker(s) present in a body fluid sample obtained from the subject with the amount of nucleosome biomarker(s) present in a body fluid sample obtained from a normal subject. It will be understood that a “normal” subject refers to a healthy/non-diseased subject.

In one embodiment, the method described herein is repeated on multiple occasions. This embodiment provides the advantage of allowing the detection results to be monitored over a time period. Such an arrangement will provide the benefit of monitoring or assessing the efficacy of treatment of a disease state. Such monitoring methods of the invention can be used to monitor onset, progression, stabilisation, amelioration, relapse and/or remission.

Thus, the invention also provides a method of monitoring efficacy of a therapy for a disease state in a subject, suspected of having such a disease, comprising detecting and/or quantifying the biomarker (e.g. nucleosome biomarkers described herein) present in a biological sample from said subject. In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the nature or amount of the biomarker(s) in test samples taken on different occasions.

Thus, according to a further aspect of the invention, there is provided a method for monitoring efficacy of therapy for a disease state in a human or animal subject, comprising:
(a) quantifying the level of one or more nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3; and
(b) comparing the result with one or more control(s) and/or one or more previous test sample(s) taken at an earlier time from the same test subject.

A change in the biomarker result in the test sample relative to the level in a previous test sample taken earlier from the same test subject may be indicative of a beneficial effect, e.g. stabilisation or improvement, of said therapy on the disorder or suspected disorder. Furthermore, once treatment has been completed, the method of the invention may be periodically repeated in order to monitor for the recurrence of a disease.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

In a further embodiment, the monitoring of more rapid changes due to fast acting therapies may be conducted at shorter intervals of hours or days.

### Kits and Panel Tests

The combination of biomarkers described herein may be used to prepare a kit or panel test, in particular for use in the diagnosis of blood cancer and/or monitoring of patients with blood cancer or suspected blood cancer.

Therefore, according to a further aspect of the invention, there is provided a kit comprising reagents to detect two or more nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3. In particular, the reagents may be used to detect the level/concentration of two or more of the listed nucleosome biomarkers. As discussed herein, three or more, four or more, five or more, six or more, seven or more, eight or more, nine or more, or all of the nucleosome biomarkers may be detected.

In a further embodiment, the kit detects the nucleosome biomarkers in a body fluid, in particular a blood, serum or plasma sample. According to a further aspect of the invention, there is provided a kit comprising reagents to measure the level of one or more (in particular, two or more) nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3, in a blood, serum or plasma sample.

According to a further aspect of the invention there is provided the use of the kit as defined herein to identify a patient in need of treatment for blood cancer.

According to a further aspect of the invention there is provided the use of the kit as defined herein to monitor a patient for progression of blood cancer. Embodiments of this aspect include use to detect disease progression in watchful waiting, active surveillance and monitoring post-surgery or other treatment for relapse.

According to a further aspect of the invention there is provided the use of the kit as defined herein to evaluate the effectiveness of a blood cancer treatment in a patient.

According to a further aspect of the invention there is provided the use of the kit as defined herein to select a treatment for a patient with blood cancer.

### Measurement Methods

In one embodiment, the level or concentration of the one or more nucleosome biomarkers (i.e. H3K9Ac, H3K9Me, H3K14Ac, H3K18Ac, H3K23Ac, H3K27Me2, H3K27Me3, H3K36Me, H3K36Me2 and H3K36Me3) detected is compared to a control. It will be clear to those skilled in the art that the control subjects may be selected on a variety of basis which may include, for example, subjects known to be free of the disease or may be subjects with a different disease (for example, for the investigation of differential diagnosis). The “control” may comprise a healthy subject, a non-diseased subject and/or a subject without cancer. Comparison with a control is well known in the field of diagnostics.

It will be understood that it is not necessary to measure healthy/non-diseased controls for comparative purposes on every occasion because once the ‘normal range’ is established it can be used as a benchmark for all subsequent tests. A normal range can be established by obtaining samples from multiple control subjects without cancer and testing for the level of biomarker. Results (i.e. biomarker levels) for subjects suspected to have cancer can then be examined to see if they fall within, or outside of, the respective normal range. Use of a ‘normal range’ is standard practice for the detection of disease.

If a subject is determined to not have cancer, then the invention may still be used for the purposes of monitoring disease progression. For example, if the use comprises a blood, serum or plasma sample from a subject determined not to have cancer, then the biomarker level measurements can be repeated at another time point to establish if the biomarker level has changed.

References to “subject” or “patient” are used interchangeably herein. In one embodiment, the patient is a human patient. In one embodiment, the patient is a (non-human) animal. The use, panels and methods described herein are preferably performed in vitro.

In one embodiment, detection or measurement of one or more of said biomarkers comprises an assay, such as an immunoassay, immunochemical, mass spectroscopy, chromatographic, chromatin immunoprecipitation or biosensor method.

In one embodiment, the detection or measurement comprises an immunoassay. In a preferred embodiment of the invention there is provided a 2-site immunoassay method for nucleosome moieties. In particular, such a method is preferred for the measurement of nucleosomes or nucleosome incorporated epigenetic features in situ employing two anti-nucleosome binding agents or an anti-nucleosome binding agent in combination with an anti-histone modification or anti-histone variant or anti-DNA modification or anti-adducted protein detection binding agent. In another embodiment of the invention, there is provided a 2-site immunoassay employing a labelled anti-nucleosome detection binding agent in combination with an immobilized anti-histone modification or anti-histone variant or anti-DNA modification or anti-adducted protein binding agent.

Detecting or measuring the level of the biomarker(s) may be performed using one or more reagents, such as a suitable binding agent. In one embodiment, the one or more binding agents comprises a ligand or binder specific for (i.e. it specifically binds to) the desired biomarker, e.g. H3K9Ac, H3K9Me, H3K14Ac, H3K18Ac, H3K23Ac, H3K27Me2 and/or H3K36Me2, or a structural/shape mimic of the biomarker or component part thereof. The term “biomarker” as defined herein includes any single biomarker moiety or a combination of individual biomarker moieties in a biomarker panel.

In preferred embodiments, the binding agent is an antibody. In one embodiment, the assay employs a single binding agent. In another embodiment, the immunoassay is a 2-site immunometric (or sandwich) assay employing two binding agents, such as antibodies. As described herein, the antibodies or other binding agents may be directed to bind to any epitope present in a nucleosome including without limitation binding to a histone, nucleosome core or DNA epitope. In some embodiments in which nucleosome adducts are measured, one or more antibodies may be directed to bind to a protein adducted to a nucleosome.

It will be clear to those skilled in the art that the terms “antibody”, “binder” or “ligand” in regard to any aspect of the invention is not limiting but intended to include any binder capable of binding to particular molecules or entities and that any suitable binder can be used in the method of the invention.

Methods of detecting biomarkers are known in the art. In one embodiment, the reagents comprise one or more ligands or binders. In one embodiment, the ligands or binders of the invention include naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

Diagnostic or monitoring kits (or panels) are provided for performing methods of the invention. Such kits will suitably comprise one or more ligands for detection and/or quantification of the biomarker according to the invention, and/or a biosensor, and/or an array as described herein, optionally together with instructions for use of the kit.

A further aspect of the invention is a kit for detecting the presence of a disease state, comprising a biosensor capable of detecting and/or quantifying one or more of the biomarkers as defined herein. As used herein, the term “biosensor” means anything capable of detecting the presence of the biomarker. Examples of biosensors are described herein. Biosensors may comprise a ligand binder or ligands, as described herein, capable of specific binding to the biomarker. Such biosensors are useful in detecting and/or quantifying a biomarker of the invention.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace. Biosensors to detect one or more biomarkers of the invention include acoustic, plasmon n resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers of the invention.

Biomarkers for detecting the presence of a disease are essential targets for discovery of novel targets and drug molecules that retard or halt progression of the disorder. As the result for a biomarker or biomarker panel is indicative of disorder and of drug response, the biomarker is useful for identification of novel therapeutic compounds in in vitro and/or in vivo assays. Biomarkers and biomarker panels of the invention can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide directed to a biomarker according to the invention; or the use of a biosensor, or an array, or a kit according to the invention, to identify a substance capable of promoting and/or of suppressing the generation of the biomarker.

The term “biomarker” means a distinctive biological or biologically derived indicator of a process, event, or condition. Biomarkers can be used in methods of diagnosis, e.g. clinical screening, and prognosis assessment and in monitoring the results of therapy, identifying subjects most likely to respond to a particular therapeutic treatment, drug screening and development. Biomarkers and uses thereof are valuable for identification of new drug treatments and for discovery of new targets for drug treatment.

The term “detecting” or “diagnosing” as used herein encompasses identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

Identifying and/or quantifying can be performed by any method suitable to identify the presence and/or amount of a specific protein in a biological sample from a subject or a purification or extract of a biological sample or a dilution thereof. In methods of the invention, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner.

Identification and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying can be performed using an immunological method, such as an immunoassay. Immunoassays include any method employing one or more antibodies or other specific binders directed to bind to the biomarkers defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels. All of said immunoassay methods are well known in the art, see for example Salgame et al. (1997) and van Nieuwenhuijze et al. (2003).

In another example, detecting and/or quantifying can be performed by one or more method(s) selected from the group consisting of: SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving identification and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Suitable biosensors for performing methods of the invention include “credit” cards with optical or acoustic readers. Biosensors can be configured to allow the data collected to be electronically transmitted to the physician for interpretation and thus can form the basis for e-medicine.

The identification of biomarkers for a disease state permits integration of diagnostic procedures and therapeutic regimes. Detection of a biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus by monitoring a biomarker of the invention, subject care can be tailored precisely to match the needs determined by the disorder and the pharmacogenomic profile of the subject, the biomarker can thus be used to titrate the optimal dose, predict a positive therapeutic response and identify those subjects at high risk of severe side effects.

Biomarker-based tests provide a first line assessment of ‘new’ subjects, and provide objective measures for accurate and rapid diagnosis, not achievable using the current measures.

Biomarker monitoring methods, biosensors and kits are also vital as subject monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

It will be understood that the embodiments described herein may be applied to all aspects of the invention, i.e. the embodiment described for the uses may equally apply to the claimed methods and so forth.

The invention will now be illustrated with reference to the following non-limiting examples.

### EXAMPLES

### Example 1—Methods

### Mass Spectrometry Protocol

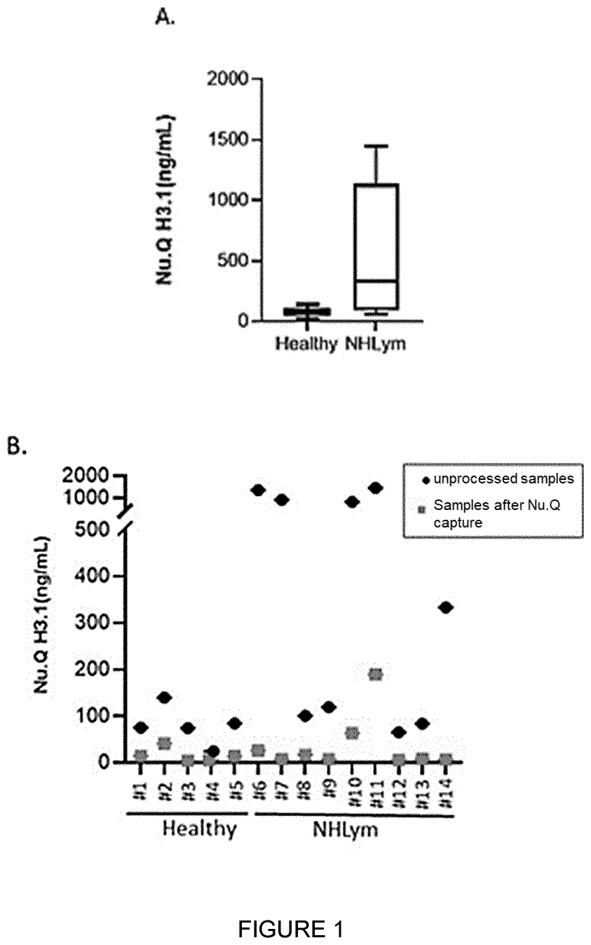

Samples were analysed using a mass spectrometry protocol described in Van den Ackerveken et al. (2021). Briefly, 900 μl of plasma samples containing circulating nucleosomes were incubated with anti-H3.1 antibody coated magnetic beads to isolate nucleosomes captured from the rest of plasma. This step provided enrichment of nucleosomes from patient samples (results shown in FIG. 1). Chemical derivatization of histones by acylation was then used to block the lysine residues and generate compatible peptides for Liquid Chromatography-Mass Spectrometry (LC-MS) analysis. After trypsin digestion, heavy amino acid labeled histone H3 peptides were added during sample preparation to each sample. These synthetic histone peptides are used for normalization to eliminate potential bias caused by sample preparation or instrumentation. Desalted peptides were then injected into a High-Performance Liquid Chromatography (HPLC) system (ULTIMATE 3000 RSLCnano System, Thermo Fisher Scientific). The eluent from the HPLC was directly electro-sprayed into a Q Exactive HF mass spectrometer (Thermo Fisher Scientific). The mass spectrometer was operated in MS/MS acquisition mode. Then, the raw peptides intensities were normalized using the intensities of spiked-in corresponding heavy peptides for H3 derived peptides. For the normalization of peptides without spiked-in heavy standards (e.g., from H2A/B and H4), the normalization was done using the overall intensity trend of the heavy standards. The log 2 of these normalized intensities are used for subsequent comparisons between experimental groups.

To arrive at the total amount of a detected modification at a particular position on a histone, it is possible to sum all peptides that hold this modification and calculate a ratio. Considering H3 positions K9 & K14 as an example, to accurately represent the amounts of each modification at those positions we can report each peptide fragment that holds these modifications. Peptide fragment KSTGGKAPR (SEQ ID NO. 1; K9 and K14 underlined, respectively) may be unmodified, methylated (Me), di-methylated (Me2) or tri-methylated (Me3) at each lysine position resulting in 16 possible combinations. Therefore the ratio of, for example, K9Me relative to all other modifications may be calculated as:

$$\frac{[ \begin{matrix}( K 9 MeK {14}_{area} ) + ( K 9 MeK 14 {Me}_{area} ) + \\ ( K 9 MeK 14 Me {2}_{area} ) + ( K 9 MeK 14 Me {3}_{area} )\end{matrix} ]}{( all peptides identified by mass {spectrometry}_{area} )} \times 100 \%$$

The reference to ‘area’ refers to the area under the relevant peak identified by mass spectrometry (which reflects the intensity/abundance of the related peptide). This form of ratiometric analysis allows for samples to be compared even if modest differences in histone recovery exist because the ratios are intra-sample calculations.

cfDNA Extraction

QIAamp® DSP Circulating NA kit (Qiagen; Antwerpen—Belgium) was used to extract the circulating DNA from the plasma samples following manufacturer's instructions. Briefly, the experiment consists of lysing, binding, washing and elution of cfDNA from the plasma samples by using QIAamp Mini columns on a vacuum manifold. In this context, at least 500 μL of plasma sample was extracted and a minimum of 30 μL of elution buffer was used to provide a suitable concentration of cfDNA for the following analysis. The amount of DNA was then normalized by multiplying the concentration obtained with the Qubit (ng/μl) by the elution buffer volume used (μl) and divided by the volume of the sample extracted (ml) to obtain a concentration expressed as ng of cfDNA per ml of plasma.

The quantification of the circulating double stranded DNA (dsDNA) was measured by the Qubit dsDNA High Sensitivity (Life Technologies Europe B.V; Merelbeke—Belgium) with a Qubit® 4.0 Fluorometer (Life Technologies Europe B.V; Merelbeke—Belgium) according to the manufacturer's instructions. The range of quantification is 0.1-120 ng. The DNA concentration was normalized upon the volume of plasma extracted and expressed as ng/mL of plasma.

The cfDNA sizing of the plasma sample was assessed by using an Agilent 2100 Bioanalyzer (Agilent Technologies; Diegem—Belgium) with an Agilent High Sensitivity DNA Kit for fragment sizes of 50-7000 bp (Agilent Technologies; Diegem—Belgium), according to manufacturer's instructions. Briefly, the experiment consists of electrophoretically separating nucleic acid fragments through interconnected microchannels based on their size.

### Quantification of Histone PTMs and Histone Variants Using an Chemiluminescent Immunoassay (Nu.Q® ChLIA Assay)

7 nucleosome structures were assessed using Nu.Q® Immunoassays (Belgian Volition SRL, Isnes, Belgium) according to the manufacturer's instructions. These sandwich immunoassays are performed on the IDS-i10 automated chemiluminescence immunoanalyzer system (Immunodiagnostic Systems Ltd (IDS) UK) using magnetic bead technology. Briefly, 50 μL of K2-EDTA plasma were mixed and incubated with an acridinium ester labeled anti-nucleosome antibody (called detection antibody). Then the anti-histone modification/variant coated beads were incubated with the complex nucleosome-detection antibody. Finally, after a wash step, the chemiluminescent substrate is added and the signal emitted was measured by the luminometer system. The results are expressed in RLU (Relative light Unit) and the concentrations were evaluated using a four-parameter logistic regression of a reference standard curve. If the % CV between the determined concentration of the duplicate measurements was above 20%, the sample was repeated once.

### Statistical Analysis

The heat map and box and whisker plots showing the min to max distribution and PCA (Principal Component Analysis) statistical analysis were performed using GraphPad InStat software (GraphPadSoftware, USA) using Log 2 of normalized ratios (defined as ratios of relative abundances normalized over the average value of the given PTM across all samples). Mann-Whitney test was used for unpaired comparisons and one-way ANOVA Test was used for multiple comparisons (*: p-value<0.05; **: p<0.01; ***: p<0.001).

### Example 2—Analysis of Non-Hodgkin Lymphoma (NHL) Samples

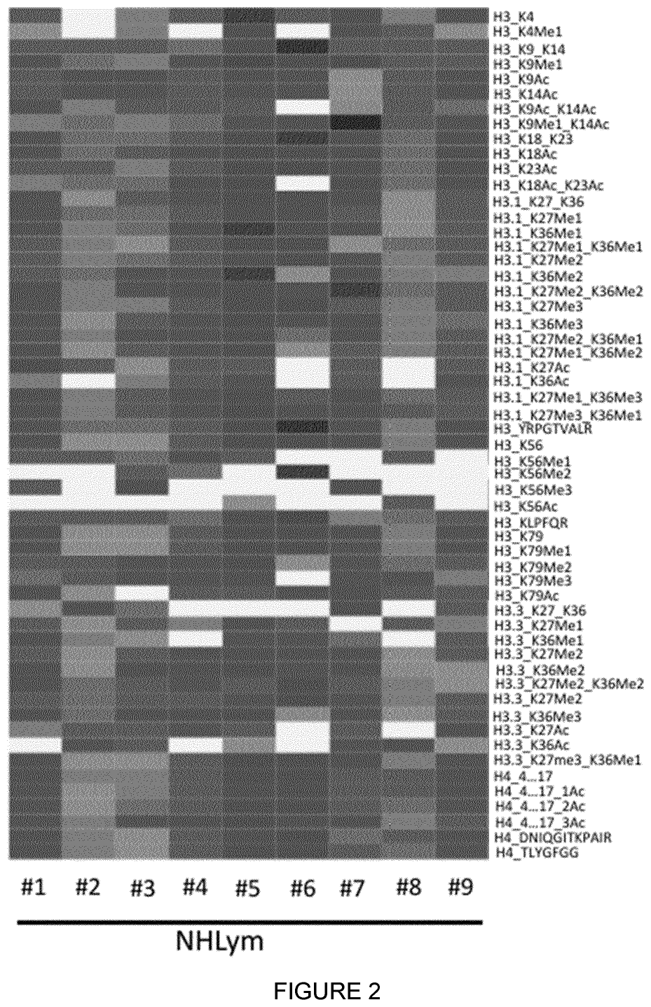

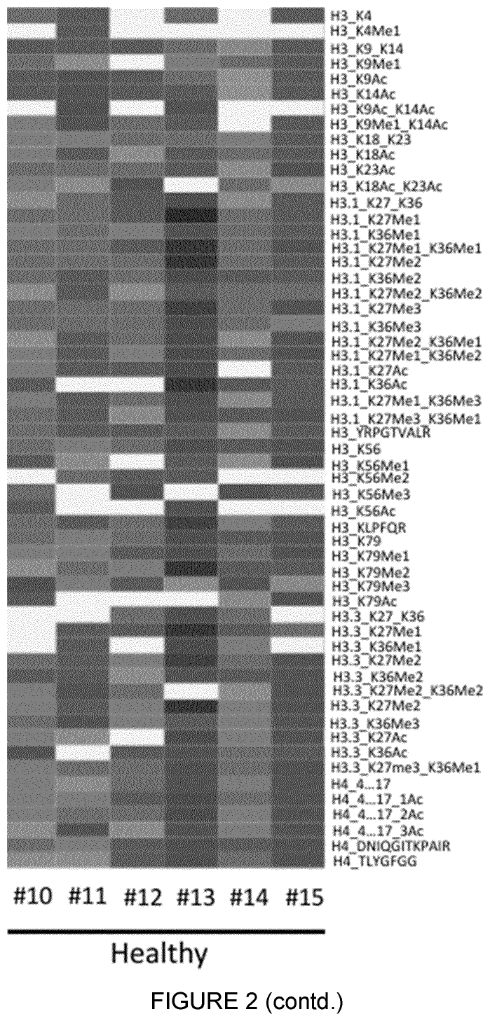

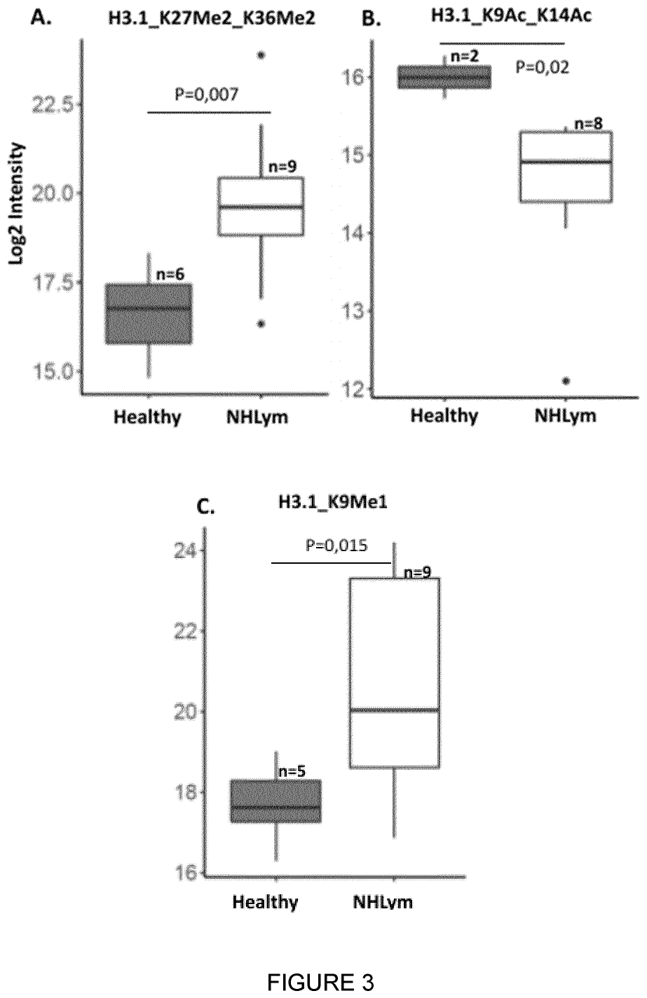

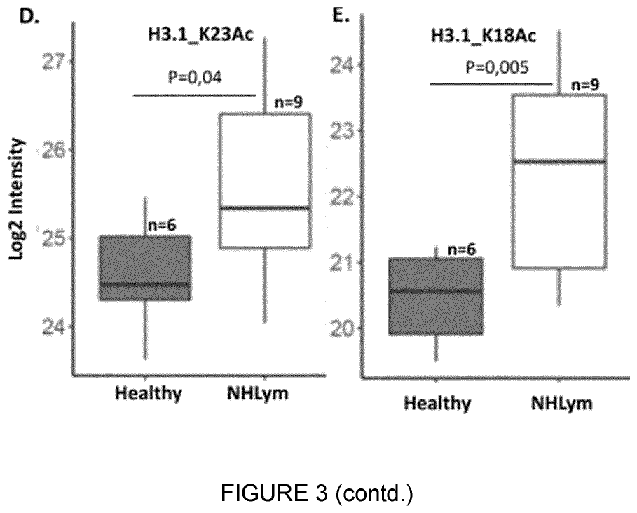

We implemented the mass spectrometry method described in Example 1 in a study composed by plasma samples from NHL cancer patients (n=9) and healthy controls (n=6). By comparing NHL vs healthy plasma samples after the protocol, we identified 56 histone proteoforms (FIG. 2). Core histone proteins such as histone H3 and H4 were identified in all immunoprecipitated samples confirming the immunoprecipitation of circulating nucleosomes. Among the histone peptides identified, we found 5 histone peptides differentially represented in plasma from NHL patients or healthy donors (p<0.05), allowing the characterization of at 7 histone PTMs located at 7 different sites (FIG. 3). The protocol was therefore effective at isolating circulating nucleosomes from plasma samples of NHL patients to allow their subsequent analysis by mass spectrometry and the discovery of biomarkers for NHL cancer diagnosis.

### Example 3—H3.1-Nucleosomes are Associated with Cell-Free Circulating DNA in NHL Patient Samples

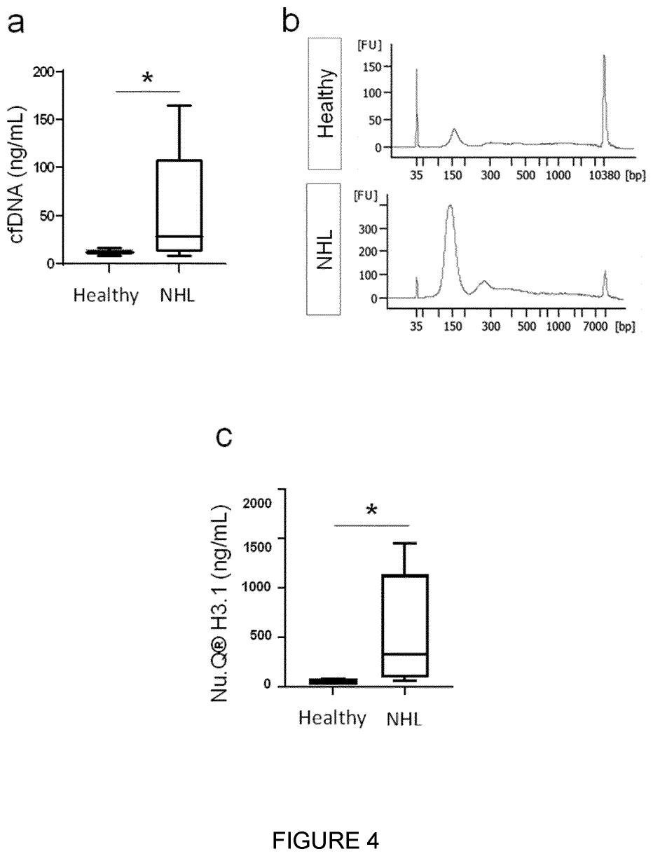

The level of H3.1-nucleosomes in blood was evaluated on K2-EDTA plasma samples from 9 NHL patients and 5 healthy donors by Nu.Q® H3.1 immunoassay. Results showed a higher level of circulating H3.1-nucleosomes in NHL patients (mean: 582.3±564.4, n=9 patients) in comparison with healthy donors (mean: 56.18 ng/mL±29.28, n=5 donors, p_value=0.012) (FIG. 4(c)). We then assessed the level of cell free DNA (cfDNA) from plasma of the same NHL patients and healthy donors. For that purpose, the cfDNA from NHL patients (n=7/9) and healthy donors (n=5) was extracted and quantified using the Qubit DNA quantification assays. We detected an increase of cfDNA levels in patients suffering from NHL (mean=53.68±59.73 ng/ml) in comparison to healthy donors (mean=12.32±2.823 ng/ml, p=0.037) (FIG. 4(a)). We then measured the strength of the relationship between these two variables using a Spearman's correlation analysis. The Spearman's correlation coefficient value obtained (r: 0.762, p=0.006) highlights the significant strong association between cfDNA concentration and the amount of H3.1-nucleosomes. To support this result, we performed a fragment size analysis of the cfDNA extracted from NHL patients using the Agilent 2100 Bioanalyzer (FIG. 4(b)). The electropherogram results showed that the average cfDNA fragment size in NHL patients was 157.7 bp±7.653 bp (range 147 bp-168 bp) corresponding to the length of DNA wrapped around a single nucleosome (˜160 bp) with sometimes a dinucleosome DNA length (˜280 bp). Altogether these results suggest that cfDNA is mainly bound to H3.1-nucleosomes in plasma K2-EDTA samples of NHL patients.

### Example 4—Mass Spectrometry Allows the Epigenetic Profile Analysis of Circulating Nucleosomes of NHL Patients

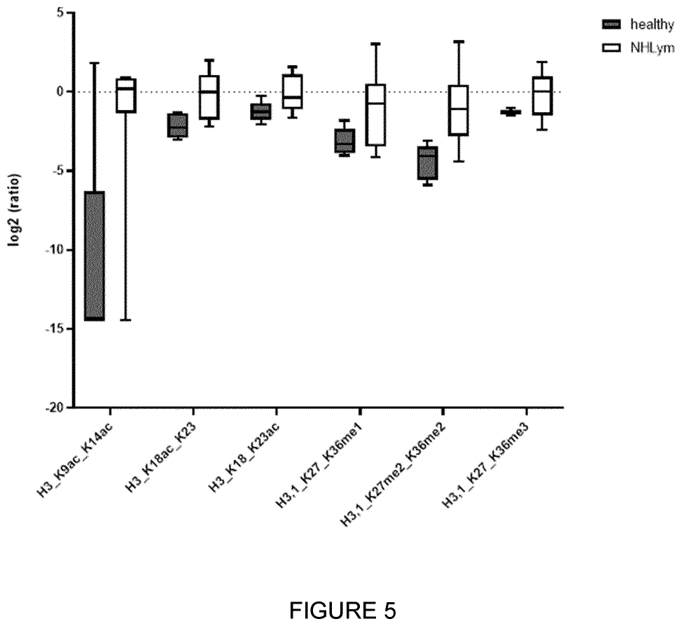

We applied the Mass spectrometry protocol described in Example 1 on a clinical cohort composed by K2-EDTA plasma samples of NHL patients (n=9) and healthy donors (n=5). To highlight the histone PTMs dysregulated in NHL blood, we analysed the results based on the log 2 of ratios, defined as ratios of relative abundances normalized over the average value of the given PTM across all samples. Among the histone peptides identified, we found 6 histone proteoforms differentially represented in plasma from NHL patients or healthy donors (p<0.05) allowing the characterization of 8 distinct histone PTMs located at 6 different sites (FIG. 5). Notably, we observed a hyperacetylation of some lysines of the histone H3 (H3K9Ac, H3K14Ac, H3K18Ac and H3K23Ac) as well as an elevation of the methylation of the histone H3 (H3K36Me1/2/3 and H3K27Me2). To further examine the relationships between the 8 distinct histone PTMs significantly dysregulated and the health status (i.e. NHL vs healthy), we performed a principal component analysis (PCA). The variance explanation chart indicates that the two first principal components (PC) are responsible for about 89.82% of the variance (PC1: 75.69% and PC2: 14.13%). Interestingly those two PCs revealed differences between subjects with the separation of most of the healthy donors to the NHL patients (data not shown). Altogether, these results suggest that the Mass spectrometry protocol allows identification of histone PTM patterns that could be used to separate NHL patients from healthy donors.

### Example 5—Further Analysis of Post-Translational Modifications of the Histone H3 Identified by Mass Spectrometry

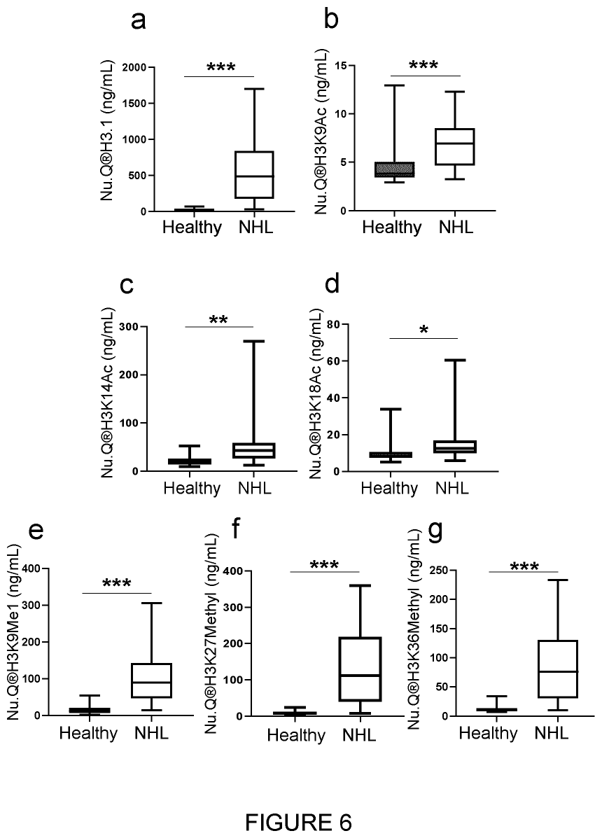

For 7 out of 8 histone PTMs identified, quantitative Nu.Q® immunoassays were available. Therefore, we decided to confirm the mass spectrometry results in an independent cohort composed by 24 NHL patients and 35 healthy donors using K2-EDTA quantitative immunoassays targeting either H3.1 or H3K9Ac or H3K9Me or H3K14Ac or H3K18Ac or H3K27Me3 (referred to as “H3K27Methyl”) or H3K36Me3 (referred to as “H3K36Methyl”)-nucleosomes. The level of H3.1-nucleosome concentration is drastically increased in NHL patients compared to healthy donors. The median reached 483.6 ng/ml in NHL patients (range 28.8 ng/ml-1699 ng/ml, n=22) versus 12.11 ng/ml for healthy donors (range 4.3 ng/ml-67.47 ng/ml, n=35) (p<0.001; FIG. 6 (a)). Moreover, the NHL patients have significantly higher levels of histone H3 acetylation (median, H3K9Ac=6.93 ng/ml; H3K14Ac=42.99 ng/ml; H3K18Ac=12.40 ng/ml; FIG. 6 (b, c, d)) and methylation (median; H3K9Me1=90.03 ng/ml; H3K27Me3=112.09 ng/ml; H3K36Me3=76.27 ng/ml; FIG. 6 (e, f, g)) when compared with healthy controls median (H3K9Ac=3.83 ng/ml; H3K14Ac=18.67 ng/mL; H3K18Ac=8.15 ng/ml; H3K9Me1=13.97 ng/ml; H3K27Me3=8.79 ng/ml; H3K36Me3=10.14 ng/ml; p<0.05). A significant positive Spearman's correlation was observed between all histone PTMs tested, therefore the immunoassay results confirmed the mis-regulation of the histone PTMs discovered by mass spectrometry. Altogether, these immunoassay results confirmed the elevation of H3.1-circulating nucleosome and the mis-regulation of the histone PTMs discovered by mass spectrometry.

In this study, we describe for the first time a high level of circulating H3.1-nucleosomes in human plasma of NHL patients associated with an elevated concentration of cfDNA. Subsequently, the nucleosome enrichment combined with mass spectrometry profiling allowed us to decipher a distinct epigenetic pattern of circulating nucleosomes in NHL. Furthermore, the clinical validation of the histone PTM pattern using immunoassays identified histones PTMs which are significantly dysregulated in NHL patients in comparison with healthy donors. The application of this histone PTM pattern on NHL patients highlights the potentially powerful use of these new biomarkers in liquid biopsy to evaluate therapeutic response and monitor tumour progression. Liquid biopsy offers a new promising diagnosis tool for cancer detection as it is safer and cheaper than tissue biopsy.

### REFERENCES

Herranz and Esteller, Methods Mol Biol, 361:25-62, 2007
Holdenrieder et al, Int J Cancer, 95:114-20, 2001
Holdenrieder and Stieber, Crit Rev Clin Lab Sci, 46 (1): 1-24, 2009
Salgame et al, Nucleic Acids Res, 25 (3): 680-681, 1997
van Nieuwenhuijze et al, Ann Rheum Dis, 62:10-14, 2003
Van den Ackerveken et al, Sci Rep, 11:7256, 2021
Zhao and Garcia, Cold Spring Harb Perspect Biol, 7: a025064, 2015

1. A method for diagnosing or monitoring blood cancer, comprising:
detecting in a body fluid sample from a subject a level of one or more histone modifications selected from the group consisting of H3K9Ac, H3K9Me, H3K14Ac, H3K18Ac, H3K23 Ac, H3K27Me2, H3K27Me3, H3K36Me, H3K36Me2 and H3K36Me3; and
using the level detected in the body fluid sample to determine if the subject has blood cancer.

2. The method of claim 1, which comprises a panel of two or more of said histone modifications.

3. The method of claim 2, wherein the histone modifications comprise H3K9Ac and H3K14Ac; or H3K27Me2 and H3K36Me2.

4. The method of claim 1, wherein the one or more histone modifications are selected from the group consisting of H3K9Ac, H3K9Me, H3K14Ac, H3K18Ac, H3K23Ac, H3K27Me3 and H3K36Me3.

5. The method of claim 1, wherein the body fluid sample is a blood, serum or plasma sample.

6. The method of claim 1, wherein the blood cancer is Non-Hodgkin Lymphoma (NHL).

7. (canceled)

8. A method of treating blood cancer in a patient, comprising;
(i) detecting or measuring a level of one or more nucleosomes selected from the group consisting of H3K9Ac, H3K9Me, H3K14Ac, H3K18Ac, H3K23Ac, H3K27Me2, H3K27Me3, H3K36Me, H3K36Me2 and H3K36Me3, in a body fluid sample obtained from the patient;
(ii) using the level detected in the body fluid sample to determine if the patient has blood cancer; and
(iii) administering a treatment to the patient if the patient is determined to have blood cancer in step (ii).

9. The method of claim 8, wherein the one or more nucleosomes are selected from the group consisting of H3K9Ac, H3K9Me, H3K14Ac, H3K18Ac, H3K23 Ac, H3K27Me3 and H3K36Me3.

10. The method of claim 8, wherein step (i) comprises measuring the level of H3K9Ac, H3K9Me, H3K14Ac, H3K18Ac, H3K23Ac, H3K27Me3 and H3K36Me3.

11. The method of claim 8, further comprising determining at least one clinical parameter for the patient.

12. (canceled)

13. The method of claim 8, further comprising measuring, in the body fluid sample, cell free DNA (cfDNA), nucleosomes per se or an epigenetic feature of a cell free nucleosome.

14. The method of claim 13, wherein the epigenetic feature is a histone isoform.

15. The method of claim 14, wherein the histone isoform is H3.1.

16-17. (canceled)

18. The method of claim 8, wherein the blood cancer is Non-Hodgkin Lymphoma (NHL).

19. The method of claim 8, wherein the detecting or measuring is performed using a technique selected from the group consisting of an immunoassay, an immunochemical assay, mass spectroscopy, a chromatographic assay, chromatin immunoprecipitation and a biosensor.

20. The method of claim 19, wherein the immunoassay employs a single binding agent.

21. The method of claim 19, wherein the immunoassay is a 2-site immunometric assay employing two binding agents.

22. The method of claim 20, wherein the binding agent is an antibody.

23. The method of claim 8, wherein the patient is a human or an animal subject.

24. A kit comprising reagents to detect two or more nucleosome biomarkers selected from the group consisting of: H3K9Ac, H3K9Me, H3K14Ac, H3K18Ac, H3K23Ac, H3K27Me2, H3K27Me3, H3K36Me, H3K36Me2 and H3K36Me3.

25-26. (canceled)


SEQUENCE LISTING (1 sequence)

SEQ ID NO: 1 - protein, 9 aa, synthetic construct
KSTGGKAPR